(19) United States
(12) Patent Application Publication
(10) Pub. No.: US 2019/0000031 A1
(43) Pub. Date: Jan. 3, 2019

(54) IMPROVED VARIETY OF SORGHUM

(71) Applicant: National University Corporation Nagoya University, Nagoya-shi, Aichi (JP)

(72) Inventors: Takashi SAZUKA, Nagoya-shi, Aichi (JP); Shigemitsu KASUGA, Nagano (JP)

(21) Appl. No.: 16/070,972

(22) PCT Filed: Jan. 16, 2017
(86) PCT No.: PCT/JP2017/001175

§ 371 Date: Jul. 18, 2018

(30) Foreign Application Priority Data
Jan. 19, 2016 (JP) 2016-008121

(51) Int. Cl. A01H 6/46; C12Q 1/6895; A01H 5/10

(52) U.S. Cl. CPC A01H 6/4666; C12Q 1/6895; C12Q 2600/16; C12Q 2600/13; A01H 5/10

(57) ABSTRACT

An object of the present invention is to provide a sorghum variety that has higher biomass, and contains a high proportion of sugar raw material (i.e., a higher sugar content). The present invention provides an improved plant derived from sorghum variety 74LH3213, wherein chromosome 6 DNA is partially or entirely substituted by a DNA region comprising a high-sugar-content locus qBRX-6 derived from sorghum variety SIL-05; and an improved Tentaka variety obtained by crossing the improved 74LH3213 plant with sorghum variety MS79A.

### TECHNICAL FIELD

The present invention relates to an improved sorghum plant.

### BACKGROUND ART

The development of biorefinery technology is progressing under the philosophy of “sustainable development.” Biorefinery technology is a technique of producing biofuels, resins, etc., by using biological resources as starting materials. As specific examples of biorefinery technology, techniques comprising obtaining sugar raw material, such as glucose or sucrose, by using a herbaceous or woody plant body as a starting material, and then obtaining the following from the sugar raw material are known: biofuels such as bioethanol; various resins such as commodity plastics, engineering plastics, cellulose materials, and composite materials; pharmaceutical intermediates; food additives; and the like. Such technology is based on obtaining sugar raw material from biomass. Enhancing this efficiency is an important issue.

Sugarcane, which has a high biomass and can provide sugar raw material by a simple treatment (such as juice-squeezing), is useful as a starting material for biorefinery technology. However, since sugarcane is also a raw material of edible sugar, extended use of biorefinery technology problematically increases the cost of edible sugar. Therefore, the development of a starting material for biorefinery technology that can replace sugarcane is crucially important.

Some sorghum varieties (Sorghum bicolor) have a high biomass, similar to that of sugarcane. For example, a sorghum variety called Tentaka has an average culm length of about 3.5 m, which is equivalent to the level of sugarcane. However, Tentaka does not contain a high proportion of sugar raw material. Therefore, obtaining sugar raw material from Tentaka in a highly efficient manner cannot be expected. In contrast, a sorghum variety called SIL-05 contains a high proportion of sugar raw material, but has a low biomass.

### CITATION LIST

### Non-Patent Literature

NPL 1: New Agricultural Genome Project (issued by: Agriculture, Forestry and Fisheries Research Council Secretariat, Ministry of Agriculture, Forestry, and Fisheries), No. 512, pp. 1-253, issued in March 2014
NPL 2: Yonemaru J, Ando T, Mizubayashi T, Kasuga S, Matsumoto T, Yano M. Development of genome-wide simple sequence repeat markers using whole-genome shotgun sequences of sorghum (Sorghum bicolor (L.) Moench). DNARes. 2009 June; 16(3): 187-193.

### SUMMARY OF INVENTION

### Technical Problem

An object of the present invention is to provide a sorghum variety that has a higher biomass and that contains a higher proportion of sugar raw material (a high sugar content).

### Solution to Problem

To achieve the above object, the present inventors focused on genome breeding of a high-biomass variety, Tentaka. However, Tentaka is an F₁ hybrid variety obtained by crossing sorghum variety 74LH3213 with sorghum variety MS79. There are few examples of successful genome breeding of F₁ varieties, inclusive of crops other than sorghum. Further, with respect to sorghum varieties, even a pure-line variety that is obtained by genome breeding does not exist, at least in Japan.

To obtain a variety with a high sugar content by genome breeding of Tentaka, a method, for example, of introducing qBRX-6, which is a high-sugar-content locus of high-sugar-content variety SIL-05 (Non-patent Literature (NPL) 1), by breeding using DNA markers may be considered. However, no DNA markers that can distinguish Tentaka and its parental varieties (74LH3213 and MS79) from SIL-05 were developed.

Under such difficult conditions with no precedent, and while lacking the necessary tools, the present inventors conducted extensive research. First, the present inventors selected and developed a DNA marker that can distinguish 74LH3213 and SIL-05, i.e., the parental varieties of Tentaka. The inventors further conducted extensive research, and introduced the high-sugar-content locus qBRX-6 into 74LH3213 by backcrossing using this DNA marker as an indicator to obtain an improved 74LH3213 variety. This improved 74LH3213 variety is hybridized with MS79 to obtain an improved Tentaka variety. The improved Tentaka variety had a higher sugar yield than Tentaka, and was also unexpectedly heavier than Tentaka in terms of the total single-plant weight. As a result of further research based on these findings, the present invention has been accomplished.

More specifically, the present invention includes the following embodiments:

Item 1. An improved 74LH3213 plant, which is an improved plant of sorghum variety 74LH3213, wherein chromosome 6 DNA is partially or entirely substituted by a DNA region comprising a high-sugar-content locus qBRX-6, which is derived from sorghum variety SIL-05.
Item 2. The improved 74LH3213 plant according to Item 1, wherein the DNA region is a DNA region ranging from
(A) DNA marker SB3412 to
(J) DNA marker SB3762.
Item 3. The improved 74LH3213 plant according to Item 1 or 2, wherein the substitution is present on both of a pair of chromosomes 6.
Item 4. A seed of the improved 74LH3213 plant according to any one of Items 1 to 3.
Item 5. A method for producing the improved 74LH3213 plant according to any one of Items 1 to 3, comprising the following steps (a) to (d):
(a) crossing sorghum variety 74LH3213 with sorghum variety SIL-05 to obtain an F₁ individual;
(b) backcrossing the F₁ individual to sorghum variety 74LH3213 to obtain a BC1F₁ individual (BC stands for backcrossing; BC1 indicates that the number of backcrossings is 1);
(c) repeating backcrossing several times, as necessary; and
(d) confirming that the chromosome 6 DNA in an individual obtained by at least one step selected from the group consisting of steps (a) to (c) is partially or entirely substituted by a DNA region comprising a high-sugar-content locus qBRX-6, which is derived from sorghum variety SIL-05.
Item 6. An improved Tentaka plant, which is an improved plant of sorghum variety Tentaka, the improved Tentaka plant being obtained by crossing the improved 74LH3213 plant according to any one of Items 1 to 3 with sorghum variety MS79, and the chromosome 6 DNA derived from the improved 74LH3213 plant being partially or entirely substituted by a DNA region comprising a high-sugar-content locus qBRX-6, which is derived from sorghum variety SIL-05.
Item 7. The improved Tentaka plant according to Item 6, wherein the DNA region is a DNA region ranging from
(A) DNA marker SB3412 to
(J) DNA marker SB3762.
Item 8. A seed of the improved Tentaka plant according to Item 6 or 7.
Item 9. A method for producing the improved Tentaka plant according to any one of Items 6 to 8, comprising crossing the improved 74LH3213 plant according to any one of Items 1 to 3 with sorghum variety MS79.
Item 10. A kit for distinguishing between genomic DNA of sorghum variety SIL-05 and genomic DNA of sorghum variety 74LH3213, comprising at least one set of primers for amplifying DNA markers selected from the group consisting of the following (a) to (j):
(a) a set of primers for amplifying DNA marker SB3412,
(b) a set of primers for amplifying DNA marker SB3420,
(c) a set of primers for amplifying DNA marker 38139115_EcoRI,
(d) a set of primers for amplifying DNA marker 40436197_XhoI,
(e) a set of primers for amplifying DNA marker 40470717_BamHI,
(f) a set of primers for amplifying DNA marker SB3517,
(g) a set of primers for amplifying DNA marker SB3613,
(h) a set of primers for amplifying DNA marker SB3621,
(i) a set of primers for amplifying DNA marker SB3683, and
(j) a set of primers for amplifying DNA marker SB3762.
Item 11. The kit according to Item 10 comprising at least one set of primers for amplifying DNA markers selected from the group consisting of the above (c) to (e).
Item 12. A method for distinguishing between genomic DNA of sorghum variety SIL-05 and genomic DNA of sorghum variety 74LH3213, the method comprising using the kit according to Item 10 or 11.

### Advantageous Effects of Invention

According to the present invention, a sorghum variety (improved Tentaka plant) that has a higher biomass and that contains a high proportion of sugar raw material (a high sugar content), and the parental variety thereof (improved 74LH3213 plant) that can easily produce this variety, can be provided. Since sugar raw material can be obtained from the improved Tentaka plant in a highly efficient and simple manner, this plant can be advantageously used as a starting material for biorefinery technology. By using the DNA markers found in the present invention, further genome breeding is feasible.

### BRIEF DESCRIPTION OF DRAWINGS

FIG. 1 shows the single-plant sugar yield of each of the improved Tentaka plant (Enryu), Tentaka, and SIL-05. The unit on the ordinate is grams per plant (g/plant).

FIG. 2 shows the total single-plant weight of each of the improved Tentaka plant (Enryu), Tentaka, and SIL-05. The unit on the ordinate is gram (g).

FIG. 3 shows the Brix value of each of the improved Tentaka plant (Enryu), Tentaka, and SIL-05.

FIG. 4 shows the dry matter content of each of the improved Tentaka plant (Enryu), Tentaka, and SIL-05.

FIG. 5 shows the length of the main culm of each of the improved Tentaka plant (Enryu), Tentaka, and SIL-05. The unit on the ordinate is millimeter (mm).

FIG. 6 shows the number of days from sowing to flowering of each of the improved Tentaka plant (Enryu), Tentaka, and SIL-05.

### DESCRIPTION OF EMBODIMENTS

In the present specification, the term “comprise” encompasses the concepts of “containing,” “substantially consisting of,” and “consisting only of.”

### 1. Improved 74LH3213 Plant

The present invention relates to an improved plant of sorghum variety 74LH3213, wherein chromosome 6 DNA is partially or entirely substituted by a DNA region comprising a high-sugar-content locus qBRX-6, which is derived from sorghum variety SIL-05 (herein sometimes referred to as “improved 74LH3213 plant”). This improved plant is described below.

Sorghum variety 74LH3213 (74LH3213) is a pollen parent of sorghum variety Tentaka, which is a very early maturing sorghum. This variety is used in a variety of academic research, such as that shown in Non-patent Literature (NPL) 2; and its seeds can be obtained from, for example, The National Institute of Agrobiological Sciences. This variety was introduced into Japan from the International Center of Improvement of Corn and Wheat (CIMMYT: “El Centro Internacional de Mejoramiento de Maíz y Trigo”) in Mexico.

Sorghum variety SIL-05 is a variety with a high sugar content, and that was registered with the Ministry of Agriculture, Forestry, and Fisheries (registration number: 21454; registration date: Feb. 21, 2012). The characteristics of sorghum variety SIL-05 are summarized as follows. The heading stage occurs slightly late; green color intensity of the lamina at the heading stage is slightly pale; the costa of the flag leaf has a slight discoloration at the heading stage; green color intensity of the costa of the flag leaf (when no discoloration occurs at the heading stage) is paler; the costa of the flag leaf has slight yellowing at the heading stage; formation of awns on lemma at the flowering stage is moderate; the stigma has no coloration, or slight coloration, with anthocyanin at the flowering stage; yellowing of the stigma at the flowering stage is moderate; grain density of spikes at the end of the flowering stage is moderate; plant height is considerably high at the maturing stage; the culm has a slightly large diameter; the leaf width at the maturing stage is moderate; the spike density at the maturing stage is moderate; spikes have a narrow egg shape at the maturing stage; the head of spikes is slightly long at the maturing stage; the glume has a black color at the maturing stage; the caryopsis has a yellowish-white color after grain threshing; and the grains are ellipsoidal as viewed from the front (a RHS color chart is shown). As compared with the control variety “Hiromidori,” sorghum variety SIL-05 has, for example, the following distinguishing features: the caryopsis after grain threshing has a more yellowish-white color; ¾ of the endosperm (the longitudinal section) is vitreous; and the vitreous material of the endosperm has a light-yellow color. As compared with the control variety “KCS105,” sorghum variety SIL-05 has, for example, the following distinguishing features: the thousand-grain weight is light; and ¾ of the endosperm (the longitudinal section) is vitreous.

The improved 74LH3213 plant may be a growing plant body itself, or a part of a plant body obtained by separation from the plant body. In the present invention, the improved plant includes seeds that grow into the plant body (i.e., seeds of the improved 74LH3213 plant).

Non-patent Literature (NPL) 1 etc. reveal that the high-sugar-content locus qBRX-6 is a gene locus that imparts a high sugar content to sorghum variety SIL-05.

The DNA region comprising the high-sugar-content locus qBRX-6 derived from sorghum variety SIL-05 is not particularly limited, as long as it is a DNA region comprising the qBRX-6 contained in the chromosome 6 of sorghum variety SIL-05. The DNA region is preferably a DNA region ranging from:

(A) DNA marker SB3412
to (J) DNA marker SB3762.
This can more effectively increase the total single-plant weight of the improved Tentaka plant described below, which is obtained by crossing the improved 74LH3213 plant with sorghum variety MS79.

In the improved 74LH3213 plant, the chromosome 6 DNA is partially or entirely substituted by a DNA region comprising a high-sugar-content locus qBRX-6, which is derived from sorghum variety SIL-05. That is, the improved 74LH3213 plant has genomic DNA wherein chromosome 6 DNA of sorghum variety 74LH3213 is partially or entirely substituted by a DNA region comprising a high-sugar-content locus qBRX-6, which is derived from sorghum variety SIL-05.

In the genomic DNA of the improved 74LH3213 plant, preferably at least one DNA region selected from the group consisting of a DNA region comprising SbPhyB gene (chromosome 1), a DNA region comprising SbPhyB gene (chromosome 6), and a DNA region comprising Mdr1/Dw3 gene (chromosome 7) (preferably the DNA regions on both of the pairs of chromosomes) is derived from 74LH3213; and more preferably all of these DNA regions are derived from 74LH3213. In this preferable embodiment, at least 80%, preferably 90% or more, more preferably 95% or more, still more preferably 99% or more, and even still more preferably 100% of the DNA regions other than the “part or the entirety of chromosome 6 DNA” in the genomic DNA of the improved 74LH3213 plant are preferably derived from 74LH3213.

The “part or the entirety of chromosome 6 DNA,” which is a region to be substituted, is a DNA region corresponding to the “DNA region comprising a high-sugar-content locus qBRX-6, which is derived from sorghum variety SIL-05 on chromosome 6 of sorghum variety 74LH3213.” That is, if “the DNA region comprising a high-sugar-content locus qBRX-6, which is derived from sorghum variety SIL-05,” ranges “from the above (A) DNA marker SB3412 to (J) DNA marker SB3762,” the “part or the entirety of chromosome 6 DNA,” which is a region to be substituted, also ranges from the above “(A) DNA marker SB3412 to (J) DNA marker SB3762.”

The improved 74LH3213 plant may have the “substitution” on only one of the pair of chromosomes 6 (may be a heterozygote having the “substitution”), or may have the “substitution” on both of the pair of chromosomes 6 (may be a homozygote having the “substitution”). However, when the improved 74LH3213 plant has the “substitution” on only one of the pair of chromosomes 6, and when the improved Tentaka plant described below is obtained by crossing the improved 74LH3213 plant with sorghum variety MS79, one half of the obtained individuals has the high-sugar-content locus qBRX-6 (i.e., the improved Tentaka plant), whereas the other half of the obtained individuals does not have the high-sugar-content locus qBRX-6. Therefore, from the viewpoint of efficiently obtaining the improved Tentaka plant, the improved 74LH3213 plant preferably contains the “substitution” on both of the pair of chromosomes 6.

The improved 74LH3213 plant preferably comprises, on one or both of the pair of chromosomes 6, the regions of DNA markers (A) to (J) described below that are all derived from sorghum variety SIL-05. That is, when the DNA markers (A) to (J) described below are investigated, the results preferably show that all of the DNA markers are of SIL-05 type. This can more effectively increase the total single-plant weight of the improved Tentaka plant described below, which is obtained by crossing the improved 74LH3213 plant with sorghum variety MS79. These DNA markers were found for the first time in the present invention as DNA markers that can distinguish between genomic DNA of sorghum variety 74LH3213 and genomic DNA of sorghum variety SIL-05. Among these, information on DNA markers (A) to (B) and (F) to (J) is laid open to the public in Non-patent Literature (NPL) 2.

(A) DNA marker SB3412
(B) DNA marker SB3420
(C) DNA marker 38139115_EcoRI
(D) DNA marker 40436197_XhoI
(E) DNA marker 40470717_BamHI
(F) DNA marker SB3517
(G) DNA marker SB3613
(H) DNA marker SB3621
(I) DNA marker SB3683
(J) DNA marker SB3762.

(A) The DNA marker SB3412 is a DNA marker having a (TA)3(GA)3(GC)3(CT)3(CGC)3(TAA)7(CGC)6 motif (wherein each numeral represents the number of repetitions in variety ATx623; the number of repetitions varies depending on the kind of variety) (GenBank ID: 54964339), and is located at a position about 1.8 Mb (specifically from 1841444 to 1841722) from the 5′ end of chromosome 6. This marker can be detected by, for example, using a primer set comprising a primer comprising an oligonucleotide comprising the base sequence set forth in SEQ ID NO: 1, and a primer comprising an oligonucleotide comprising the base sequence set forth in SEQ ID NO: 2. When PCR is performed using this set of primers, the length of the resulting amplified DNA fragment is 278 in the case of variety BTx623.

(B) The DNA marker SB3420 is a DNA marker having a (TA)3(AG)3(TC)18 motif (wherein each numeral represents the number of repetitions in variety ATx623; the number of repetitions varies depending on the kind of variety) (GenBank ID: 55075727), and is located at a position about 2.5 Mb (specifically from 2548519 to 2548806) from the 5′ end of chromosome 6. This marker can be detected by, for example, using a primer set comprising a primer comprising an oligonucleotide comprising the base sequence set forth in SEQ ID NO: 3, and a primer comprising an oligonucleotide comprising the base sequence set forth in SEQ ID NO: 4. When PCR is performed using this set of primers, the length of the resulting amplified DNA fragment is 287 in the case of variety BTx623.

(C) The DNA marker 38139115_EcoRI is a dCAPS marker, and is located at a position about 38.1 Mb from the 5′ end of chromosome 6. This marker can be detected by, for example, using a primer set comprising a primer comprising an oligonucleotide comprising the base sequence set forth in SEQ ID NO: 5, and a primer comprising an oligonucleotide comprising the base sequence set forth in SEQ ID NO: 6. When PCR is performed using this set of primers and the resulting amplified DNA fragment (length: 233) is cleaved with EcoRI, the length of the longer fraction is 207 in the case of variety SIL-05. In contrast, in the case of variety 74LH3213, the resulting PCR amplified DNA fragment (length: 233) cannot be cleaved with EcoRI.

(D) The DNA marker 40436197_XhoI is a dCAPS marker, and is located at a position about 40.4 Mb from the 5′ end of chromosome 6. This marker can be detected by, for example, using a primer set comprising a primer comprising an oligonucleotide comprising the base sequence set forth in SEQ ID NO: 7, and a primer comprising an oligonucleotide comprising the base sequence set forth in SEQ ID NO: 8. When PCR is performed using this set of primers, and the resulting amplified DNA fragment (length: 229) is cleaved with XhoI, the length of the longer fraction is 203 in the case of variety 74LH3213. In contrast, in the case of variety SIL-05, the obtained PCR amplified DNA fragment (length: 229) cannot be cleaved with EcoRI.

(E) The DNA marker 40470717_BamHI is a dCAPS marker, and is located at a position about 40.5 Mb from the 5′ end of chromosome 6. This marker can be detected by, for example, using a primer set comprising a primer comprising an oligonucleotide comprising the base sequence set forth in SEQ ID NO: 9, and a primer comprising an oligonucleotide comprising the base sequence set forth in SEQ ID NO: 10. When PCR is performed using this set of primers, and the resulting amplified DNA fragment (length: 229) is cleaved with BamHII, the length of the longer fraction is 203 in the case of variety SIL-05. In contrast, in the case of variety 74LH3213, the obtained PCR amplified DNA fragment (length: 229) cannot be cleaved with BamHII.

(F) The DNA marker SB3517 is a DNA marker having a (AG)9(AG)12(TA)3(TG)5(AGC)3 motif (wherein each numeral represents the number of repetitions in variety ATx623; the number of repetitions varies depending on the kind of variety) (GenBank ID: 40656077), and is located at a position about 44.7 Mb (specifically from 44671001 to 44671206) from the 5′ end of chromosome 6. This marker can be detected by, for example, using a primer set comprising a primer comprising an oligonucleotide comprising the base sequence set forth in SEQ ID NO: 11, and a primer comprising an oligonucleotide comprising the base sequence set forth in SEQ ID NO: 12. When PCR is performed using this set of primers, the length of the resulting amplified DNA fragment is 210 in the case of variety BTx623.

(G) The DNA marker SB3613 is a DNA marker having a (AC)3(AG)3(TG)5(GAA)6 motif (wherein each numeral represents the number of repetitions in variety ATx623; the number of repetitions varies depending on the kind of variety) (GenBank ID: 54713363), and is located at a position about 51.717 Mb (specifically from 51717672 to 51717949) from the 5′ end of chromosome 6. This marker can be detected by, for example, using a primer set comprising a primer comprising an oligonucleotide comprising the base sequence set forth in SEQ ID NO: 13, and a primer comprising an oligonucleotide comprising the base sequence set forth in SEQ ID NO: 14. When PCR is performed using this set of primers, the length of the resulting amplified DNA fragment is 254 in the case of variety BTx623.

(H) The DNA marker SB3621 is a DNA marker having a (AC)3(AG)19(AC)3 motif (wherein each numeral represents the number of repetitions in variety ATx623; the number varies depending on the kind of variety) (GenBank ID: 55017903), and is located at a position about 52.13 Mb (specifically from 52137810 to 52137940) from the 5′ end of chromosome 6. This marker can be detected by, for example, using a primer set comprising a primer comprising an oligonucleotide comprising the base sequence set forth in SEQ ID NO: 15 and a primer comprising an oligonucleotide comprising the base sequence set forth in SEQ ID NO: 16. When PCR is performed using this set of primers, the length of the resulting amplified DNA fragment is 130 in the case of variety BTx623.

(I) The DNA marker SB3683 is a DNA marker having a (TC)3(TCA)8 motif (wherein each numeral represents the number of repetitions in variety ATx623; the number of repetitions varies depending on the kind of variety) (GenBank ID: 54843017), and is located at a position about 55.2 Mb (specifically 55246684 to 55246927) from the 5′ end of chromosome 6. This marker can be detected by, for example, using a primer set comprising a primer comprising an oligonucleotide comprising the base sequence set forth in SEQ ID NO: 17, and a primer comprising an oligonucleotide comprising the base sequence set forth in SEQ ID NO: 18. When PCR is performed using this set of primers, the length of the resulting amplified DNA fragment is 243 in the case of variety BTx623.

(J) The DNA marker SB3762 is a DNA marker having a (AC)3(AT)3(TCA)9(CTT)3 motif (wherein each numeral represents the number of repetitions in variety ATx623; the number varies depending on the kind of variety) (GenBank ID: 55255040), and is located at a position about 58.4 Mb (specifically 58394270 to 58394479) from the 5′ end of chromosome 6. This marker can be detected by, for example, using a primer set comprising a primer comprising an oligonucleotide comprising the base sequence set forth in SEQ ID NO: 19, and a primer comprising an oligonucleotide comprising the base sequence set forth in SEQ ID NO: 20. When PCR is performed using this set of primers, the length of the resulting amplified DNA fragment is 209 in the case of variety BTx623.

The method for producing the improved 74LH3213 plant is not particularly limited. For example, DNA marker breeding by successive backcrossings can be used. Specifically, the following method can be used: an F₁ individual obtained by crossing sorghum variety 74LH3213 with sorghum variety SIL-05 is backcrossed to sorghum variety 74LH3213 while confirming that its chromosome 6 DNA is partially or entirely substituted by a DNA region comprising a high-sugar-content locus qBRX-6, which is derived from sorghum variety SIL-05. More specifically, a method comprising the following steps can be used:

(a) crossing sorghum variety 74LH3213 with sorghum variety SIL-05 to obtain an F₁ individual;
(b) backcrossing the F₁ individual to sorghum variety 74LH3213 to obtain a BC1F₁ individual (BC stands for backcrossing); BC1 indicates that the number of backcrossings is 1);
(c) repeating backcrossing several times as necessary; and
(d) confirming that the chromosome 6 DNA in an individual obtained by at least one step selected from the group consisting of steps (a) to (c) is partially or entirely substituted by a DNA region comprising a a high-sugar-content locus qBRX-6, which is derived from sorghum variety SIL-05.

The crossing and backcrossing in steps (a) to (c) can be performed by or in accordance with a known crossing method for sorghum. The method of culturing from seeds obtained by crossing can also be performed by or in accordance with known culturing methods for sorghum.

When step (c) is performed, the total number of backcrossings performed, including the number of backcrossings in step (b) (one time), is preferably 5 or more. By increasing the number of backcrossings, DNA regions other than the DNA region comprising the high-sugar-content locus qBRX-6 derived from sorghum variety SIL-05 can more closely resemble those of sorghum variety 74LH3213. This can more stably maintain Tentaka's high biomass characteristics when the obtained improved 74LH3213 plant is crossed with sorghum variety MS79.

Step (d) can be performed, for example, by determining the type(s) of DNA marker(s) (whether the marker is SIL-05 type). As such DNA markers, for example, the above DNA markers (A) to (J) can be used.

Specific procedures in step (d) are, for example, as follows. First, DNA is extracted from a plant body. The tissue used for the DNA extraction is not particularly limited. Any tissue, such as leaves, roots, or stems, can be used. To efficiently extract nucleic acids, these tissues are preferably ground using liquid nitrogen.

Extraction of DNA can be performed by a known method. The CTBA method (Murray et al., Nucleic Acids Res. 8, 4321-4325 (1980)) is preferably used. Specifically, the following steps are performed. First, a tissue is ground in liquid nitrogen. After the ground product is incubated by adding a solution of cetyl trimethyl ammonium bromide (CTAB), chloroform-isoamyl alcohol is added, and the resulting mixture is mixed well. An aqueous layer is separated and collected by centrifugation, and isopropyl alcohol is added thereto. The formed precipitates are collected by centrifugation, then dissolved by adding a buffer solution containing, for example, EDTA; and treated with RNase. After the treatment, the solvent is replaced by phenol, phenol with chloroform-isoamyl alcohol, and chloroform-isoamyl alcohol, in this order. After the substitution processing, genomic DNA is obtained by centrifugation. The extraction can be performed by using a commercially available kit (e.g., “DNeasy,” produced by QIAGEN). The extracted DNA quantity may be any amount that enables DNA marker analysis. When PCR is performed, the extracted DNA quantity may be, for example, 1 ng or more per reaction.

Subsequently, PCR amplification is performed using the extracted DNA as a template, and using a set of primers for detecting a DNA marker. The PCR amplification is not particularly limited, as long as the set of primers described above is used. The PCR amplification can be performed in a usual manner. Specifically, a fragment comprising the target DNA marker is amplified by repeating a cycle comprising denaturation of the template DNA, annealing of the primers to the template, and a primer extension reaction using a heat-resistant enzyme (Taq polymerase). With respect to the composition of the PCR reaction mixture and PCR reaction conditions (temperature cycle, number of cycles, etc.), a person skilled in the art can appropriately select and set the conditions under which PCR amplification products can be obtained with high sensitivity by PCR using the set of primers described above. A method for selecting appropriate PCR reaction conditions based on the Tₘ of primers is well known in this technical field. Such a series of PCR procedures can be performed by using a commercially available PCR kit or PCR device in accordance with the instruction manual thereof. Examples of PCR devices include GeneAmp PCR System 9700 (produced by Applied Biosystems), GeneAmp PCR System 9600 (produced by Applied Biosystems), and the like.

The detection of the PCR amplification product can be confirmed by conventional electrophoresis, such as agarose gel electrophoresis or capillary electrophoresis, DNA crossing, real-time PCR, and like methods. For example, in agarose gel electrophoresis, after staining with ethidium bromide, SYBR Green solution, or the like, an amplification product is detected as a single band. Whether the DNA marker is of SIL-05 type is determined by comparing the band of the amplification product with a control band (a band of a PCR product of the SIL-05 variety and/or a band of a PCR product of the 74LH3213 variety).

When the DNA marker is a dCAPS marker as in (C) to (E) above, a PCR amplification product is digested with an appropriate restriction enzyme, and whether the DNA marker is of SIL-05 type is determined by comparing the position of the band of the digested fragment with the position of a control band, i.e., a band of the digested fragment of a PCR product of SIL-05 variety and/or a band of the digested fragment of a PCR product of 74LH3213 variety.

The individuals obtained in steps (a) to (d) have a substitution with a DNA region containing a high-sugar-content locus qBRX-6, which is derived from sorghum variety SIL-05, on only one of chromosome 6. Further, individuals having this substitution on both of the pair of chromosomes 6 can be obtained by obtaining self-propagating seeds of this individual.

### 2. Improved Tentaka Plant

The present invention relates to an improved Tentaka plant, which is an improved plant of sorghum variety Tentaka and is obtained by crossing an improved 74LH3213 plant with sorghum variety MS79A; and wherein the chromosome 6 DNA derived from the improved 74LH3213 plant is partially or entirely substituted by a DNA region containing a high-sugar-content locus qBRX-6, which is derived from sorghum variety SIL-05 (hereinafter referred to as “improved Tentaka plant”). This plant is described below.

Sorghum variety Tentaka is a variety with a high sugar content, and that was registered with the Ministry of Agriculture, Forestry, and Fisheries (registration number: 4290; registration date: Mar. 9, 1995). The characteristics of sorghum variety Tentaka are summarized as follows. Tentaka is a hybridized variety that has as its maternal line a cytoplasmic male sterile progeny introduced from Hiroshima Prefectural Agriculture Test Center, and that has as its paternal line an inbred progeny introduced from the National Grassland Research Institute. Tentaka is a variety suitable for fodder use; Tentaka has a very long culm, and heading does not occur under usual culture conditions. The culm is very long in length and considerably large in diameter, and has a considerably small number of tillers. The main culm has an extremely large number of leaves, with the leaves being long and wide, and with moderate leaf color intensity and leaf insertion angle. The midrib of the leaf blade has a light-green color. Tentaka is an annual plant; the early growth is moderate; the heading period, flowering period, and maturing period are very late; the stem has a mixture of juiciness and dryness, and has low sweetness; and the stems and leaves have a low dry-matter content. Tentaka has a slightly low resistance to lodging, and is highly resistant to sheath blight disease and bacterial leaf streak disease. As compared with “FS902,” Tentaka has, for example, the following distinguishing features: a large number of leaves are on the main culm; and its heading period, flowering period, and maturing period are late.

Sorghum variety MS79A (MS79A, I.S.2830A) is a seed parent of sorghum variety Tentaka, and is a cytoplasmic male sterile progeny of very early grain sorghum. This variety can be obtained, for example, in the form of seeds from the National Institute of Agrobiological Sciences.

The improved Tentaka plant may be a growing plant body itself, or a part of a plant body separated from the plant body. The present invention includes seeds that grow into the plant (i.e., seeds of the improved Tentaka plant).

In the improved Tentaka plant, chromosome 6 DNA is partially or entirely substituted by a DNA region comprising a high-sugar-content locus qBRX-6, which is derived from sorghum variety SIL-05. The meaning of the terms, preferable embodiments, etc., are the same as disclosed above in section “1. Improved 74LH3213 Plant.”

In the genomic DNA of the improved Tentaka plant, 80% or more, preferably 90% or more, more preferably 95% or more, still more preferably 99% or more, and even more preferably 100% of the DNA regions other than the “part or the entirety of chromosome 6 DNA” are preferably derived from Tentaka.

The improved Tentaka plant can be obtained by crossing an improved 74LH3213 plant with sorghum variety MS79A. When the improved 74LH3213 plant has the above substitution (substitution with a DNA region comprising the high-sugar-content locus qBRX-6) on only one of the pair of chromosomes 6, one half of the obtained individuals has the high-sugar-content locus qBRX-6 (i.e., the improved Tentaka plant described below), but the other half of the obtained individuals does not have the high-sugar-content locus qBRX-6. Accordingly, in this case, by selecting the individuals having the high-sugar-content locus qBRX-6 according to the DNA marker types disclosed above in section “I. Improved 1.74LH3213 Plant,” the improved Tentaka plant can be obtained. When the improved 74LH3213 plant contains the above substitution (substitution with a DNA region comprising the high-sugar-content locus qBRX-6) on both of the pair of chromosomes 6, the obtained individuals are all improved Tentaka plants. The crossing, determination of DNA marker types, etc. are the same as disclosed above in section “1. Improved 74LH3213 Plant.”

### 3. Kit for Discriminating Sorghum Variety

The present invention relates to a kit for distinguishing between genomic DNA of sorghum variety SIL-05 and genomic DNA of sorghum variety 74LH3213 (in this specification, sometimes referred to as “kit for discriminating a sorghum variety”) comprising at least one set of primers for amplifying DNA markers selected from the group consisting of the following (a) to (j):

(a) a set of primers for amplifying DNA marker SB3412,
(b) a set of primers for amplifying DNA marker SB3420,
(c) a set of primers for amplifying DNA marker 38139115_EcoRI,
(d) a set of primers for amplifying DNA marker 40436197_XhoI,
(e) a set of primers for amplifying DNA marker 40470717_BamHI,
(f) a set of primers for amplifying DNA marker SB3517,
(g) a set of primers for amplifying DNA marker SB3613,
(h) a set of primers for amplifying DNA marker SB3621,
(i) a set of primers for amplifying DNA marker SB3683, and
(j) a set of primers for amplifying DNA marker SB3762.

(a) There is no particular limitation on the base sequences of the oligonucleotides of each of the sets of primers (a) to (j). For example, as long as the oligonucleotides have substantially the same function as oligonucleotides comprising the base sequences set forth in SEQ ID NOs: 1 to 20, oligonucleotides comprising base sequences of SEQ ID NOs: 1 to 20 wherein one or more (for example, five, preferably three, more preferably one) bases are deleted, added, or substituted may be used.

The oligonucleotides of each of the sets of primers (a) to (j) can be synthesized by a method that is known in this technical field as a method for synthesizing an oligonucleotide, such as the phosphotriethyl method or the phosphodiester method, using a DNA automatic synthesizer commonly used (e.g., Model 394, produced by Applied Biosystems).

To facilitate the detection of PCR amplification products obtained using such primers, oligonucleotides labeled with a marker substance may be used. Examples of such marker substances include fluorescent substances well known in this technical field (e.g., FITC, ROC, etc.); radioisotopes; chemiluminescent substances (e.g., DNP); biotin; DIG (digoxigenin); and the like.

The kit for discriminating a sorghum variety may be any kit comprising at least one set of primers for amplifying DNA markers selected from the group consisting of the above (a) to (j). The kit of the present invention may further contain, as necessary, DNA extraction reagents; PCR reagents such as PCR buffer and DNA polymerase; a DNA solution containing a PCR amplified region that is used as a positive control in the reaction; detection reagents such as staining agents and electrophoresis gels; instruction manuals; and the like.

### Examples

The present invention is described in detail below with reference to Examples. However, the invention is not limited to these Examples.

### (1) Selection of DNA Markers

DNA markers capable of distinguishing between genomic DNA of sorghum variety 74LH3213 and genomic DNA of sorghum variety SIL-05 were selected from candidate DNA markers of SB markers (total: 830 sets) among the DNA markers disclosed in Non-patent Literature (NPL) 2. Specifically, to detect candidate DNA markers, PCR was performed using primer sets disclosed in Non-patent Literature (NPL) 1 and using genomic DNA sequence of sorghum variety 74LH3213 and genomic DNA sequence of sorghum variety SIL-05 as template DNAs. The presence or absence of the amplified DNA fragment and difference in length of the amplified DNA fragment between sorghum variety 74LH3213 and sorghum variety SIL-05 were investigated.

As a result, the following DNA markers (A) to (B) and (F) to (J) were found to clearly discriminate between sorghum variety 74LH3213 and sorghum variety SIL-05 in length of the amplified DNA fragment.

(A) DNA marker SB3412
(B) DNA marker SB3420
(F) DNA marker SB3517
(G) DNA marker SB3613
(H) DNA marker SB3621
(I) DNA marker SB3683
(J) DNA marker SB3762.

### (2) Production of Novel DNA Marker

The genomic DNA sequences of sorghum variety 74LH3213 and sorghum variety SIL-05 were sequenced using a next-generation sequencer. Based on this result, SNPs different between these varieties were identified. Further, dCAPS markers capable of detecting the SNP were prepared. The dCAPS markers produced are the following markers (C) to (E).

(C) DNA marker 38139115_EcoRI
(D) DNA marker 40436197_XhoI
(E) DNA marker 40470717_BamHI.

### (3) Production of Improved Plant of Sorghum Variety 74LH3213

Using the above “(1) Selection of DNA Marker” and “(2) Production of New DNA Marker,” an improved plant of sorghum variety 74LH3213 (BC5F₁-qBRX) was produced. Specifically, the improved plant was prepared as follows.

Sorghum variety 74LH3213 was crossed with sorghum variety SIL-05 to obtain an F₁ plants.

The F₁ was crossed with sorghum variety 74LH3213 to obtain a BC1F₁ plants.

The BC1F₁ was crossed with sorghum variety 74LH3213 to obtain a BC2F₁ plants. Five individuals in the BC2F₁ group were subjected to genotyping using DNA markers SB3517, SB3613, SB3683, and SB3762. One individual (BC2F₁-qBRX), in which all of these DNA marker regions on one of the pair of chromosomes 6 were derived from sorghum variety SIL-05, was selected.

The BC2F₁-qBRX was crossed with sorghum variety 74LH3213 to obtain a BC3F₁ group. Four individuals in the BC3F₁ plants were subjected to genotyping using DNA markers SB3412, SB3420, SB3517, SB3613, SB3683, and SB3762. One individual (BC3 F₁-qBRX), in which all of these DNA marker regions on one of the pair of chromosomes 6 were derived from sorghum variety SIL-05, was selected.

The BC3 F₁-qBRX was crossed with sorghum variety 74LH3213 to obtain a BC4 F₁ group. Fifteen individuals in the BC4F₁ plants were subjected to genotyping using DNA markers SB3412, SB3420, 38139115_EcoRI, 40436197_XhoI, 40470717_BamHI, SB3517, SB3613, SB3683, and SB3762. One individual (BC4F₁-qBRX) in which all of these DNA marker regions on one of the pair of chromosomes 6 were derived from sorghum variety SIL-05 was selected. Further, with respect to the BC4F₁-qBRX, using DNA markers other than those on chromosome 6 (a total of 36 sets), the chromosome DNAs other than those on chromosome 6 were confirmed to be derived from sorghum variety 74LH3213 by backcrossing.

The BC4F₁-qBRX was crossed with sorghum variety 74LH3213 to obtain a BC5F₁ group. Twenty-five individuals in the BC5F₁ plants were subjected to genotyping using DNA markers SB3412, SB3420, SB3517, SB3613, SB3621, SB3683, and SB3762. One individual (BC5F₁-qBRX), in which all of these DNA marker regions on one of the pair of chromosomes 6 were derived from sorghum variety SIL-05, was selected.

The BC5F₁-qBRX was self-propagated to obtain a BC5F₂ plants. Forty individuals in the BC5 F₂ group were subjected to genotyping using the DNA markers SB3412, SB3420, 40436197_XhoI, 40470717_BamHI, SB3517, SB3613, SB3621, SB3683, and SB3762. One individual (BC5F₂-qBRX), in which all of these DNA marker regions on one of the pair of chromosomes 6 were derived from sorghum variety SIL-05, was selected.

A variety registration application was filed to register the selected individual (BC5F₂-qBRX) as an improved plant of sorghum variety 74LH3213, under the name of “Improved 74HL No. 0)” (Ministry of Agriculture, Forestry and Fisheries of Japan; Variety Registration Application No.: 30854; Application Filing Date: Feb. 22, 2016; Applicant: Nagoya University).

### (4) Production of Improved Plant of Sorghum Variety Tentaka, and Evaluation of Properties

The BC5F₂-qBRX obtained above in section “(3) Production of Improved Plant of Sorghum Variety 74LH3213” was crossed with sorghum variety MS79A to obtain an improved plant of sorghum variety Tentaka. This improved Tentaka plant (Enryu) was collected 30 days after flowering, and the total single-plant weight, Brix value, dry-matter content, length of the main culm, and number of days from sowing to flowering were determined. Further, the single-plant sugar yield was calculated, based on these measurements.

The Brix value was calculated in the following manner. The internode located at the center of the entire culm length was sampled to obtain a roughly 1-cm internode piece as a culm sample from its intermediate site. A squeezed juice was collected from the sample using pliers. The sugar content of this squeezed juice was measured using a refractive saccharimeter (ASONE Pocket Refractometer PAL-1).

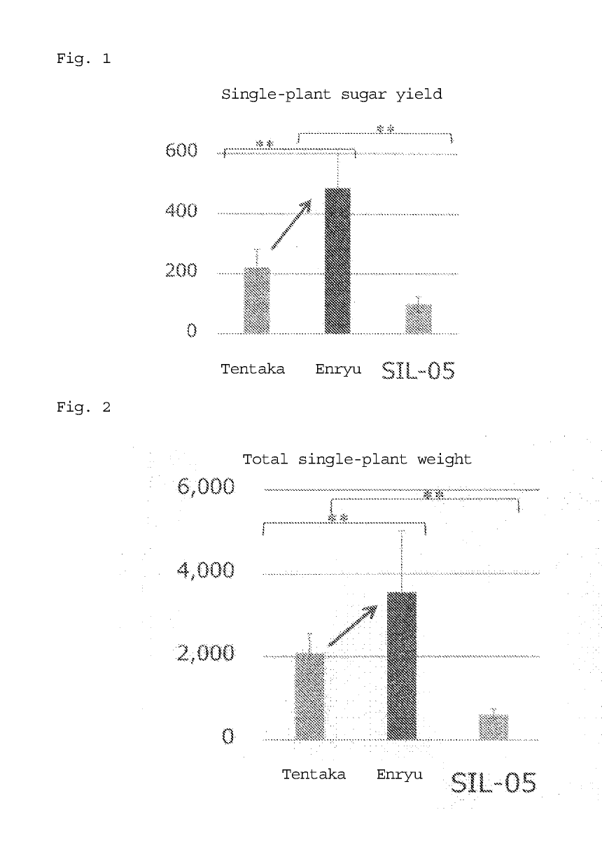

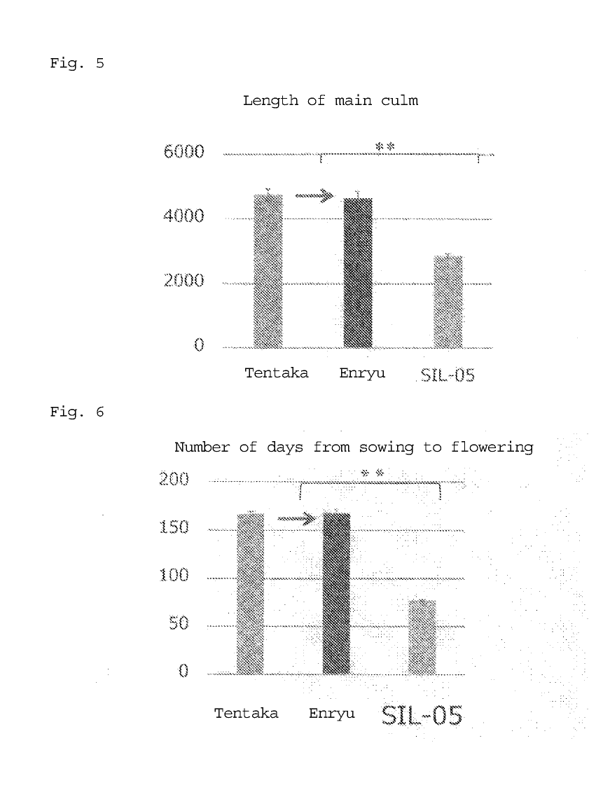

FIGS. 1 to 6 show the results. In the Figures, * indicates that the P value is 0.05 or less, and ** indicates that the P value is 0.01 or less.

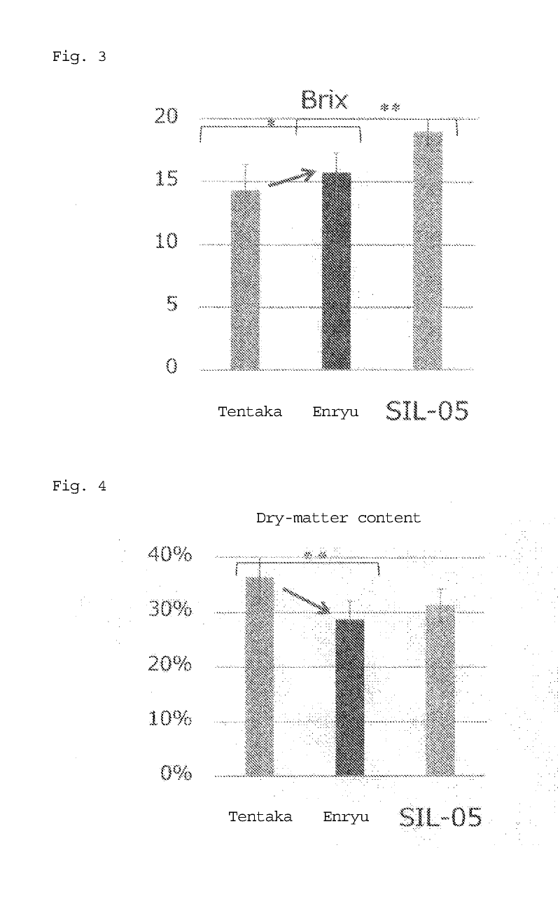

As shown in FIGS. 2 and 3, the total single-plant weight and Brix value of Enryu were higher than those of Tentaka. On the other hand, as shown in FIG. 4, the dry-matter content of Enryu was lower than that of Tentaka. This indicates that the obtained improved 76LH3213 plant was juicier. Reflecting the above results, the single-plant sugar yield of Enryu was roughly at least two times or more that of Tentaka.

1. An improved 74LH3213 plant, which is an improved plant of sorghum variety 74LH3213, wherein chromosome 6 DNA is partially or entirely substituted by a DNA region comprising a high-sugar-content locus qBRX-6, which is derived from sorghum variety SIL-05, and the DNA region is a DNA region ranging from
(A) DNA marker SB3412 to
(J) DNA marker SB3762.

2. (canceled)

3. The improved 74LH3213 plant according to claim 1, wherein the substitution is present on both of a pair of chromosomes 6.

4. A seed of the improved 74LH3213 plant according to claim 1.

5. A method for producing the improved 74LH3213 plant according to claim 1, comprising the following steps (a) to (d):
(a) crossing sorghum variety 74LH3213 with sorghum variety SIL-05 to obtain an F1 individual;
(b) backcrossing the F1 individual to sorghum variety 74LH3213 to obtain a BCH individual (BC stands for backcrossing; BC1 indicates that the number of backcrossings is 1);
(c) repeating backcrossing several times, as necessary; and
(d) confirming that the chromosome 6 DNA in an individual obtained by at least one step selected from the group consisting of steps (a) to (c) is partially or entirely substituted by a DNA region comprising a high-sugar-content locus qBRX-6, which is derived from sorghum variety SIL-05.

6. An improved Tentaka plant, which is an improved plant of sorghum variety Tentaka, the improved Tentaka plant being obtained by crossing the improved 74LH3213 plant according to claim 1 with sorghum variety MS79, the chromosome 6 DNA derived from the improved 74LH3213 plant being partially or entirely substituted by a DNA region comprising a high-sugar-content locus qBRX-6, which is derived from sorghum variety SIL-05, and the DNA region being a DNA region ranging from (A) DNA marker SB3412 to (J) DNA marker SB3762.

7. (canceled)

8. A seed of the improved Tentaka plant according to claim 6.

9. A method for producing an improved Tentaka plant, the method comprising crossing the improved 74LH3213 plant according to claim 1 with sorghum variety MS79.

10. A kit for distinguishing between genomic DNA of sorghum variety SIL-05 and genomic DNA of sorghum variety 74LH3213, comprising at least one set of primers for amplifying DNA markers selected from the group consisting of the following (a) to (j):
(a) a set of primers for amplifying DNA marker SB3412,
(b) a set of primers for amplifying DNA marker SB3420,
(c) a set of primers for amplifying DNA marker 38139115_EcoRI,
(d) a set of primers for amplifying DNA marker 40436197_XhoI,
(e) a set of primers for amplifying DNA marker 40470717_BamHI,
(f) a set of primers for amplifying DNA marker SB3517,
(g) a set of primers for amplifying DNA marker SB3613,
(h) a set of primers for amplifying DNA marker SB3621,
(i) a set of primers for amplifying DNA marker SB3683, and
(j) a set of primers for amplifying DNA marker SB3762.

11. The kit according to claim 10 comprising at least one set of primers for amplifying DNA markers selected from the group consisting of the above (c) to (e).

12. A method for distinguishing between genomic DNA of sorghum variety SIL-05 and genomic DNA of sorghum variety 74LH3213, the method comprising using the kit according to claim 10.

13. A seed of the improved 74LH3213 plant according to claim 3.

14. A method for producing the improved 74LH3213 plant according to claim 3, comprising the following steps (a) to (d):
(a) crossing sorghum variety 74LH3213 with sorghum variety SIL-05 to obtain an F1 individual;
(b) backcrossing the F1 individual to sorghum variety 74LH3213 to obtain a BC1f individual (BC stands for backcrossing; BC1 indicates that the number of backcrossings is 1);
(c) repeating backcrossing several times, as necessary; and
(d) confirming that the chromosome 6 DNA in an individual obtained by at least one step selected from the group consisting of steps (a) to (c) is partially or entirely substituted by a DNA region comprising a high-sugar-content locus qBRX-6, which is derived from sorghum variety SIL-05.

15. An improved Tentaka plant, which is an improved plant of sorghum variety Tentaka, the improved Tentaka plant being obtained by crossing the improved 74LH3213 plant according to claim 3 with sorghum variety MS79, the chromosome 6 DNA derived from the improved 74LH3213 plant being partially or entirely substituted by a DNA region comprising a high-sugar-content locus qBRX-6, which is derived from sorghum variety SIL-05, and the DNA region being a DNA region ranging from (A) DNA marker SB3412 to (J) DNA marker SB3762.

16. A method for producing an improved Tentaka plant, the method comprising crossing the improved 74LH3213 plant according to claim 3 with sorghum variety MS79.

17. A method for distinguishing between genomic DNA of sorghum variety SIL-05 and genomic DNA of sorghum variety 74LH3213, the method comprising using the kit according to claim 11.


SEQUENCE LISTING (20 sequences)

SEQ ID NO: 1 - DNA, 24 bp, Sorghum bicolor
aatgcttttcatccatcagcaacc

SEQ ID NO: 2 - DNA, 24 bp, Sorghum bicolor
cttcgctgtgatttttctttccgt

SEQ ID NO: 3 - DNA, 24 bp, Sorghum bicolor
gagccagcatgcatgataattgtt

SEQ ID NO: 4 - DNA, 24 bp, Sorghum bicolor
cacaaaggcatgacagtcaatcaa

SEQ ID NO: 5 - DNA, 30 bp, Sorghum bicolor
gcaaacactgtcagcacaactatcagaatt

SEQ ID NO: 6 - DNA, 20 bp, Sorghum bicolor
tactttgacccctttcatac

SEQ ID NO: 7 - DNA, 30 bp, Sorghum bicolor
actatgcctctcaagccaaggagttctcga

SEQ ID NO: 8 - DNA, 20 bp, Sorghum bicolor
cttctcagtgactacatcca

SEQ ID NO: 9 - DNA, 29 bp, Sorghum bicolor
ctgcgcgccatggagccgttcccccggtc

SEQ ID NO: 10 - DNA, 20 bp, Sorghum bicolor
ggtaaccgccgaccaaattg

SEQ ID NO: 11 - DNA, 24 bp, Sorghum bicolor
cgaatcagttttgtgcatcttcgt

SEQ ID NO: 12 - DNA, 24 bp, Sorghum bicolor
agtgccggaatcctagaaagaagg

SEQ ID NO: 13 - DNA, 24 bp, Sorghum bicolor
cttgtcttctgaaacggagggaga

SEQ ID NO: 14 - DNA, 24 bp, Sorghum bicolor
gtccatctgaccgtttcatcacag

SEQ ID NO: 15 - DNA, 24 bp, Sorghum bicolor
ggttcttggcattacctgcaaaag

SEQ ID NO: 16 - DNA, 24 bp, Sorghum bicolor
gcaatttgcactggttacttgctg

SEQ ID NO: 17 - DNA, 24 bp, Sorghum bicolor
gcgaaaaggattttcaactatggg

SEQ ID NO: 18 - DNA, 24 bp, Sorghum bicolor
tttgtcttgcagtttcctgacgag

SEQ ID NO: 19 - DNA, 24 bp, Sorghum bicolor
tgggtgtcagcaatgaagacattt

SEQ ID NO: 20 - DNA, 24 bp, Sorghum bicolor
catccgtttagtttcggctttctg